(19) United States
(12) Patent Application Publication
(10) Pub. No.: US 2013/0052178 A1
(43) Pub. Date: Feb. 28, 2013

(54) CHOLESTEROL ESTER FOR REDUCING CHOLESTEROL ABSORPTION

(71) Applicants: Maria Jadwiga Bauerek, Arhus N (DK); Charlotte Horsmans Poulsen, Brabrand (DK); Jørn Borch Søe, Mundelstrup (DK); Bent Raungaard, Hojbjerg (DK); Leif Schauser, Arhus C (DK)

(72) Inventors: Maria Jadwiga Bauerek, Arhus N (DK); Charlotte Horsmans Poulsen, Brabrand (DK); Jørn Borch Søe, Mundelstrup (DK); Bent Raungaard, Hojbjerg (DK); Leif Schauser, Arhus C (DK)

(73) Assignee: DuPont Nutrition Biosciences ApS, Copenhagen K (DE)

(21) Appl. No.: 13/696,271

(22) PCT Filed: May 6, 2011
(86) PCT No.: PCT/IB2011/052009

§ 371 Date: Nov. 5, 2012

Related U.S. Application Data
(60) Provisional application No. 61/376,023, filed on Aug. 23, 2010.

(30) Foreign Application Priority Data
May 7, 2010 (GB) 1007668.5

(51) Int. Cl. A61K 31/56; A61P 3/06; C12N 9/10; A61K 38/45; C07J 9/00

(52) U.S. Cl. 424/94.5; 514/182; 552/544; 435/193

(57) ABSTRACT

The present invention relates to a method for reducing cholesterol absorption in an animal comprising administering to the animal a composition comprising an effective amount of at least one cholesterol ester.

### FIELD OF THE INVENTION

The present invention relates to methods of reducing blood cholesterol in an individual. More specifically the invention relates to methods of reducing blood cholesterol by administering at least one cholesterol ester to the individual. The present invention also relates to pharmaceutical compositions and foodstuffs comprising cholesterol esters.

### BACKGROUND

High plasma LDL-cholesterol (LDL-C) levels are associated with the development of atherosclerosis. Clinical studies show that plant sterols induce reduction in serum LDL-cholesterol concentrations in mild hypercholesterolemic subjects. Therefore dietary plant sterols are recommended as adjunctive lifestyle treatment for hypercholesterolemia. While the absorption of intraluminal cholesterol and plant sterols is well described, the effects of dietary cholesteryl esters has not been considered. The present inventors have undertaken research to identify the effect of cholesterol esters on the levels of cholesterol in the blood.

### SUMMARY

The present invention is based on the highly surprising discovery by the inventors that the presence of cholesterol esters can reduce the absorption of cholesterol by intestinal cells resulting in lower levels of blood cholesterol.

Therefore, according to a first aspect of the present invention there is provided a method for reducing cholesterol absorption in an animal comprising administering to the animal a composition comprising an effective amount of at least one cholesterol ester.

According to a second aspect of the present invention there is provided a method for reducing cholesterol absorption in an animal comprising administering to the animal a composition comprising at least one lipid acyltransferase.

According to a third aspect of the present invention there is provided a composition comprising at least one cholesterol ester for use in therapy.

According to a fourth aspect of the present invention there is provided a composition comprising at least one cholesterol ester for use in reducing the level of blood cholesterol in an individual.

According to a fifth aspect of the present invention there is provided a composition comprising at least one lipid acyltransferase for use in reducing the level of blood cholesterol in an individual.

According to a sixth aspect of the present invention there is provided a pharmaceutical composition comprising at least one cholesterol ester and at least one pharmaceutically acceptable diluent, excipient and/or carrier.

According to a seventh aspect of the present invention there is provided a foodstuff comprising at least one exogenously produced cholesterol ester.

According to an eighth aspect of the present invention there is provided the use of a cholesterol ester in the manufacture of a medicament for reducing the level of blood cholesterol in an individual.

According to an ninth aspect of the present invention there is provided the use of a lipid acyltransferase in the manufacture of a medicament for reducing the level of blood cholesterol in an individual.

According to a tenth aspect of the present invention there is provided a method for regulating cholesterol absorption in an individual not suffering from hypercholesterolemia comprising administering to the individual a composition comprising an effective amount of at least one cholesterol ester.

According to an eleventh aspect of the present invention there is provided a method for regulating cholesterol absorption in an individual not suffering from hypercholesterolemia comprising administering to the individual a composition comprising an effective amount of at least one lipid acyltransferase.

### DETAILED DESCRIPTION

In the description which follows, it will be understood that any of the preferred features described are applicable to any aspect of the present invention unless explicitly stated otherwise.

### Cholesterol Esters

It will be apparent to the skilled person that as used herein, the term cholesterol ester relates to any cholesterol ester, for example, cholesterol fatty acid esters including cholesterol fatty acid esters in which the fatty acid is saturated or unsaturated. Furthermore, the terms cholesterol ester and cholesteryl ester are used interchangeably.

In preferred embodiments the cholesterol ester has a structure as shown in Formula I:

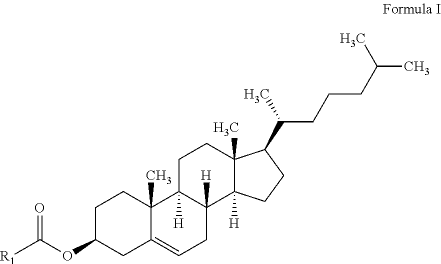

In a preferred aspect, R₁ in Formula I is a C₁-C₃₅ hydrocarbon group.

Here the term “hydrocarbon” means any one of an alkyl group, an alkenyl group, or an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

Suitable substituent(s) are hydroxyl groups. In a preferred aspect, the compound has between 0 to 3 substitients, more preferably 0 to 2, more preferably 0 or 1.

Preferably R₁ in Formula I is a C₄-C₂₄ hydrocarbon group. More preferably R₁ is a C₁₀-C₂₃ hydrocarbon group, more preferably R₁ is a C₉-C₁₇ hydrocarbon group, such as a C₁₃-C₁₇ group. In a highly preferred aspect R₁ is a C₁₇ hydrocarbon group.

In one aspect, R₁ is a hydrocarbon group comprising an alkenyl group. Preferably this hydrocarbon group comprises from 1 to 6 C=C double bonds. Preferably this hydrocarbon group comprising an alkenyl group comprises from 1 to 3 C=C double bonds.

In one aspect, R₁ is a saturated hydrocarbon group. Preferably R₁ is a (CH₂)ₙCH₃ group, wherein n is zero or a positive integer. Preferably n is an integer from 6 to 28, more preferably 8 to 22, more preferably 14 to 20, such as 14 to 18. In a highly preferred aspect n is 16.

In one preferred embodiment, the cholesterol ester for use in the present invention comprises at least one fatty acid having a carbon chain length of 10:0, 10:1, 12:0, 12:1, 13:0, 13:1, 14:0, 14:1, 15:0, 15:1, 16:0, 16:1, 17:0, 18:0, 18:1, 18:2, 20:0, 20:1, 20:2 wherein the first number relates to the fatty acid carbon chain length and the second number refers to the number of double bonds present in the carbon chain.

In a more preferred embodiment, the cholesterol ester comprises at least one of cholesteryl linoleate (C18:2), cholesteryl oleate (C18:1), cholesteryl stearate (C18:0), cholesteryl palmitate(C16:0), cholesteryl palmitoleate (C16:1), cholesteryl myristate (C14:0), cholesteryl laurate (C12:0) and/or cholesteryl caprate (C10:0). In a more preferred embodiment, the cholesterol ester comprises a mixture of at least two of the recited esters.

It will be understood that the cholesterol ester may be obtained from any suitable source, naturally occurring or synthetic. In one preferred embodiment, the cholesterol ester is enzymatically produced using a lipid acyltransferase. Preferably, the cholesterol ester is produced from at least one of egg, milk and/or meat.

It will be apparent to the skilled person that the cholesterol moiety of the cholesterol ester may be from any suitable source. Preferably, the cholesterol moiety is from an animal source.

It will be understood that the sterol moiety for use in the methods and uses of the present invention is not a plant sterol.

It will further be apparent to the skilled person that when the cholesterol ester is a cholesterol fatty acid ester, the fatty acid can be provided from any suitable source. Preferably, the fatty acid is provided by a triglyceride or phospholipid.

In one embodiment, the hydrocarbon group is provided by a plant or animal source. In one preferred embodiment, the hydrocarbon group is from dairy fat. In an alternative preferred embodiment, the hydrocarbon group is from at least one plant oil.

In a further embodiment, the hydrocarbon group is not provided from a plant source.

In one preferred embodiment of the present invention, the cholesterol ester is present in a foodstuff.

Preferably, the foodstuff is selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces. More preferably, the foodstuff is milk or a milk product.

It will be apparent to the skilled person that the cholesterol ester may be produced exogenously and added to the foodstuff and/or may be produced in situ by the action of a lipid acyltransferase. It will be further apparent that a number of foodstuffs may comprise naturally occurring cholesterol esters. However, the skilled person would understand that these naturally occurring esters are present at sub-clinical levels which have no significant effect on the absorption of cholesterol by intestinal cells. The skilled person will understand that the present invention relates to methods and compositions which comprise addition amounts of cholesterol ester beyond that which may naturally present in a foodstuff.

It will be understood that the terms produced exogenously and exogenously produced as used herein means that the lipid acyltransferase is not produced in situ, for example, in the foodstuff, but is added thereto in an appropriate amount to give the desired concentration.

The skilled person will understand that cholesterol absorption occurs primarily in the duodenum and proximal jejunum at levels of efficiency that vary greatly among different individuals. There are two main phases of cholesterol absorption, the first takes place in the lumen and involves digestion and hydrolysis of dietary lipids followed by solubilization of cholesterol in mixed micelles containing bile acid and phospholipids. This solubilization facilitates the movement of cholesterol from the bulk phase of the lumen to the surface of the enterocyte. In the second phase, cholesterol crosses the mucosal cell membrane.

As used herein the terms blood cholesterol refers to the total cholesterol level in the blood. It will be apparent to the skilled person that this includes LDL cholesterol and HDL cholesterol and VLDL cholesterol.

It will be understood that the term animal used herein refers to both humans and other types of animal, particularly mammals. It will be further understood that as used herein the term individual refers to a human or other animal.

In a preferred embodiment, the animal to be administered the compositions of the present invention is a human. In a more preferred embodiment, the animal is a human suffering from hypercholesterolemia.

In one embodiment the methods of the present invention relate to methods of treating hypercholesterolemia.

It will be understood that as used herein the term treating hypercholesterolemia refers to both treating individuals suffering from hypercholesterolemia and the prophylactic treatment of individuals at risk of developing hypercholesterolemia.

It will be apparent to a skilled person that individuals may suffer from different levels of hypercholesterolemia. In a preferred embodiment of the present invention an individual to be administered the compositions of the present invention is an individual suffering from mild to moderate hypercholesterolemia (5.2-8.0 mmol cholesterol/L blood; LDL-C in the range from about 130-159 mg/dl [mild] to about 160-219 mg/dl [moderate]).

In a further embodiment the animal or individual is an animal or individual suffering from severe hypercholesterolemia (LDL-C of greater than 220 mg/dl).

In an alternative embodiment the animal or individual is an animal or individual having a LDL-C level of less than 130 mg/dl.

In a further preferred aspect the present invention relates to a method for regulating cholesterol absorption in an individual not suffering from hypercholesterolemia comprising administering to said individual a composition comprising at least one cholesterol ester and/or at least one lipid acyltransferase.

It will be understood that administration of at least one cholesterol ester or lipid acyltransferase to an individual having normal levels of blood cholesterol may reduce or prevent an increase in the level of blood cholesterol.

It will be understood that the composition may be administered in any suitable form. Preferably the composition is suitable for oral administration. In one preferred embodiment the composition is a foodstuff. In an alternative embodiment the composition is an oral composition suitable to be taken as a food supplement.

It will be understood that an individual may be at risk of developing hypercholesterolemia due to a variety of reasons, for example, as a result of obesity, diet, familial hypercholesterolemia, type 2 diabetes, hypothyroidism, or side effects of other medication.

In a preferred aspect the present invention relates to a composition comprising at least one cholesterol ester for use in therapy. More preferably, the composition further comprises at least one lipid acyltransferase.

In a further aspect, the present invention relates to a foodstuff comprising at least one exogenously produced cholesterol ester.

In one preferred embodiment, the foodstuff further comprises at least one lipid acyltransferase. The skilled person will understand that if the foodstuff comprises a lipid acyl donor and cholesterol, further cholesterol esters as described above may be produced in situ in the foodstuff.

Preferably, the foodstuff is selected from one or more of: eggs, egg-based products, including but not limited to mayonnaise, salad dressings, sauces, ice creams, egg powder, modified egg yolk and products made there from; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

More preferably, the foodstuff is milk or a milk based product.

It will be apparent to the skilled person that in various embodiments of the aspects of the present invention, the cholesterol ester is added to the foodstuff. In alternative embodiments the cholesterol ester is not added to the foodstuff. In further embodiments, the cholesterol ester is generated in the foodstuff. In yet further embodiments, the cholesterol is not generated in the foodstuff.

The present invention also provides a pharmaceutical composition comprising at least one cholesterol ester and/or at least one lipid acyltransferase for use in the methods or uses of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Preferably the pharmaceutical composition is in a form that is suitable for oral delivery.

Preferably, the cholesterol ester is provided at a level in the foodstuff or pharmaceutical composition or food supplement to result in administration to an individual of a dosage of between about 0.001 g and about 10 g per day, about 0.01 g and about 5 g per day, about 0.1 g and about 3 g per day based on a recommended portion size in relation to food or a recommended dosage regime in relation to a pharmaceutical or food supplement.

In an alternative embodiment the cholesterol ester is provided at a dosage of less than 10 g per day, less than 7 g per day, less than 5 g per day, less than 3 g per day, less than 2 g per day, less than 1 g per day, less than 0.5 g per day, less than 0.1 g per day, less than 0.05 g per day or less than 0.01 g per day.

In an alternative embodiment the cholesterol ester is provided at a dosage of more than 0.01 g per day, more than 0.05 g per day, more than 0.1 g per day, more than 0.5 g per day, more than 1 g per day, more than 2 g per day, more than 3 g per day, more than 5 g per day, more than 7 g per day or more than 10 g per day.

In some preferred embodiments the pharmaceutical composition or food supplement is administered before, or during, or after a meal. It will be understood that the terms before and after mean within 2 hours, preferably 1 hour, more preferably 30 minutes, even more preferably 15 minutes of beginning/finishing the meal. It will be understood that in some embodiments the meal may be a high cholesterol meal.

In an alternative embodiment the pharmaceutical or food supplement is designed to be taken 1, or 2, or 3 or 4 times daily.

In a preferred embodiment, the foodstuff or pharmaceutical or food supplement comprises at least two different cholesterol esters.

### Lipid Acyltransferases

It will be apparent to the skilled person that the at least one lipid acyltransferase for use in the aspects of the present invention may be any lipid acyltransferase.

For instance, the lipid acyl transferase for use in the present invention may be one as described in WO2004/064537, WO2004/064987, WO2005/066347, WO2006/008508 or WO2008/090395. These documents are incorporated herein by reference.

The lipid acyl transferase for use in any one of the methods and/or uses of the present invention may be a natural lipid acyl transferase or a variant lipid acyl transferase.

The term “lipid acyltransferase” as used herein means an enzyme which has acyltransferase activity (for example an enzyme classified as E.C. 2.3.1.x, in particular 2.3.1.43 in accordance with the Enzyme Nomenclature Recommendations (1992) of the Nomenclature Committee of the International Union of Biochemistry and Molecular Biology), whereby the enzyme is capable of transferring an acyl group from a lipid to cholesterol.

Suitably the lipid acyltransferase is one classified under the Enzyme Nomenclature classification (E.C. 2.3.1.43). Such enzymes are commercially available from Danisco A/S and are sold under the trade name LysoMax Oil™ and Food-Pro Cleanline™.

Preferably, the lipid acyl transferase for use in any one of the methods and/or uses of the present invention is a lipid acyltransferase that is capable of transferring an acyl group from a phospholipid to a sterol.

Other acyltransferases suitable for use in the present invention include phospholipid:diacylglycerol acyltransferases from enzyme class E.C 2.3.1.158 as disclosed in WO03/100044 (incorporated herein by reference), diacylglycerol-sterol O-acyltransferases from class E.C 2.3.1.73 which catalyse the reaction 1,2-diacyl-sn-glycerol+sterol→monoacylglycerol+sterol ester, and sterol O-cyltransferases from class E.C 2.3.1.26 which catalyse the reaction acyl-CoA+cholesterol→CoA+cholesterol ester.

Suitably, the acyltransferase activity of enzymes for use in the present invention accounts for at least 5%, more preferably at least 10%, more preferably at least 20%, more preferably at least 30%, more preferably at least 40%, more preferably 50%, more preferably at least 60%, more preferably at least 70%, more preferably at least 80%, more preferably at least 90% and more preferably at least 98% of the total enzyme activity. The % transferase activity (i.e. the transferase activity as a percentage of the total enzymatic activity) may be determined by the following protocol:

### Determination of Lipid Acyltransferase Activity

Substrate: 50 mg Cholesterol (Sigma C8503) and 450 mg Soya phosphatidylcholine(PC), Avanti #441601 is dissolved in chloroform, and chloroform is evaporated at 40° C. under vacuum.

300 mg PC:cholesterol 9:1 is dispersed at 40° C. in 10 ml 50 mM HEPES buffer pH 7.

Enzymation:

250 μl substrate is added in a glass with lid at 40° C.

25 μl enzyme solution is added and incubated during agitation for 10 minutes at 40° C.

The enzyme added should esterify 2-5% of the cholesterol in the assay.

Also a blank with 25 μl water instead of enzyme solution is analysed.

After 10 minutes 5 ml Hexan:Isopropanol 3:2 is added.

The amount of cholesterol ester may be analysed by HPLC using Cholesteryl stearate (Sigma C3549) standard for calibration.

Transferase activity is calculated as the amount of cholesterol ester formation per minute under assay conditions.

One Transferase Unit (TrU) is defined as μmol cholesterol ester produced per minute at 40° C. and pH 7 in accordance with the transferase assay given above.

Preferably, the lipid acyltransferase used in the method and uses of the present invention will have a specific transferase unit (TrU) per mg enzyme of at least 25 TrU/mg enzyme protein.

Suitably the lipid acyltransferase for use in the present invention may be dosed in amount of 0.05 to 50 TrU per g phospholipid composition, suitably in an amount of 0.5 to 5 TrU per g phospholipid composition.

More preferably the enzymes suitable for use in the methods and/or uses of the present invention have lipid acyl-transferase activity as defined by the protocol below:

### Protocol for the Determination of % Acyltransferase Activity:

A foodstuff to which a lipid acyltransferase for use according to the present invention has been added may be extracted following the enzymatic reaction with CHCl₃:CH₃OH 2:1 and the organic phase containing the lipid material is isolated and analysed by GLC according to the procedure detailed herein below. From the GLC analysis or HPLC analysis the amount of free fatty acids and one or more cholesterol esters is determined. A control foodstuff to which no enzyme according to the present invention has been added, is analysed in the same way.

### Calculation:

From the results of the GLC (and optionally HPLC analyses) the increase in free fatty acids and cholesterol esters can be calculated:

Δ% fatty acid=% Fatty acid(enzyme)−% fatty acid(control);

Mv fatty acid=average molecular weight of the fatty acids;

A=Δ% cholesterol ester/Mv cholesterol ester (where Δ% cholesterol ester=% cholesterol ester(enzyme)−% cholesterol ester(control) and Mv cholesterol ester=average molecular weight of the cholesterol esters).

The transferase activity is calculated as a percentage of the total enzymatic activity:

$$\% transferase activity = \frac{A \times 100}{A + Δ \% fatty acid \text{/} ( Mv fatty acid )} .$$

GLC analysis

GLC analysis may be performed using any suitable apparatus. In this case a Perkin Elmer Autosystem 9000 Capillary Gas Chromatograph equipped with WCOT fused silica column 12.5 m×0.25 mm ID×0.1 μ film thickness 5% phenyl-methyl-silicone (CP Sil 8 CB from Chrompack) was used.

| Carrier gas: | Helium. | | |
|---|---|---|---|
| Injector. | PSSI cold split injection (initial temp 50° C. | | |
| | heated to 385° C.), volume 1.0 μl | | |
| Detector FID: | 395° C. | | |
| Oven program: | 1 | 2 | 3 |
| Oven temperature, ° C. | 90 | 280 | 350 |
| Isothermal, time, min. | 1 | 0 | 10 |
| Temperature rate, ° C./min. | 15 | 4 | |

Sample preparation: 30 mg of extracted lipid sample was dissolved in 9 ml Heptane:Pyridin, 2:1 containing internal standard heptadecane, 0.5 mg/ml. 300 μl sample solution was transferred to a crimp vial, 300 μl MSTFA (N-Methyl-N-trimethylsilyl-trifluoraceamid) was added and reacted for 20 minutes at 60° C. Calculation: Response factors for mono-di-triglycerides and free fatty acid were determined from Standard 2 (mono-di-triglyceride), for Cholesterol, Cholesteryl palmitate and Cholesteryl stearate, and the response factors were determined from pure reference material (weighing for pure material 10 mg).

It will be readily apparent to one skilled in the art that compositions comprising lipid acyltransferases for use in the present invention may further comprises at least one cholesterol ester as described above.

In one preferred embodiment, the lipid acyltransferase is present in a foodstuff.

Preferably, the foodstuff is selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces. More preferably, the foodstuff is milk or a milk product.

It will be understood that the methods and uses of the present invention can result in a reduced uptake of cholesterol and subsequent lowering of the level of blood cholesterol in an animal. It will be apparent that in cases where the animal has a low starting level of cholesterol in the blood, no or no significant lowering will be seen. However, in animals where the starting level of cholesterol in the blood is high such as in individuals exhibiting hypercholesterolemia, the reduction in the level of cholesterol will be higher. This reduction will be dependent upon the level of cholesterol ester administered with higher levels of cholesterol ester resulting in a greater reduction in cholesterol absorption by the intestinal cells. In a preferred embodiment, the cholesterol ester results in a reduction of at least 2%, 5%, 7%, 10%, 15%, 20%, 25%, 30%, 35%, 40%, 45% or 50% in the uptake of cholesterol by intestinal cells.

### EXAMPLES

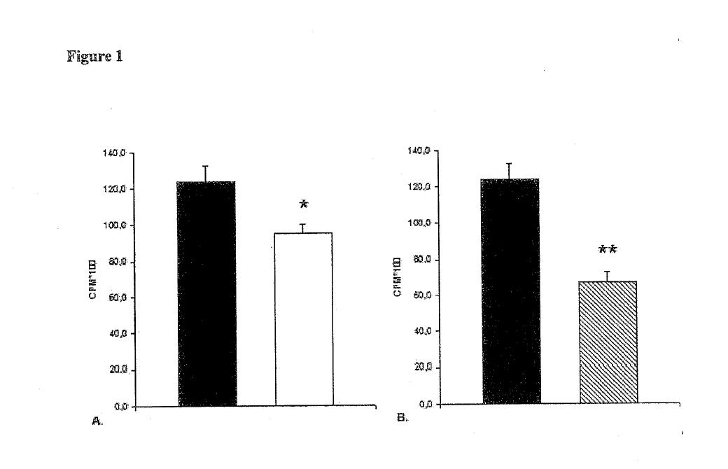

FIG. 1 shows Sterol absorption into Caco-2 cells using artificial micelles. Panel A: shows inclusion of cholesteryl oleate in cholesterol containing micelles (white column) decreased the uptake of micellar cholesterol into CaCo-2 cells compared to the uptake from micelles containing cholesterol only (black column). Panel B: shows that uptake of cholesteryl oleate by CaCo-2 cells (hatched column) was less than uptake of cholesterol (black column). Each column and vertical bar represents mean±SD. *p<0.001 for ³H-cholesterol containing micelles with inclusion of unlabeled cholesteryl oleate, **p<0.001 for ³H-cholesteryl oleate micelles

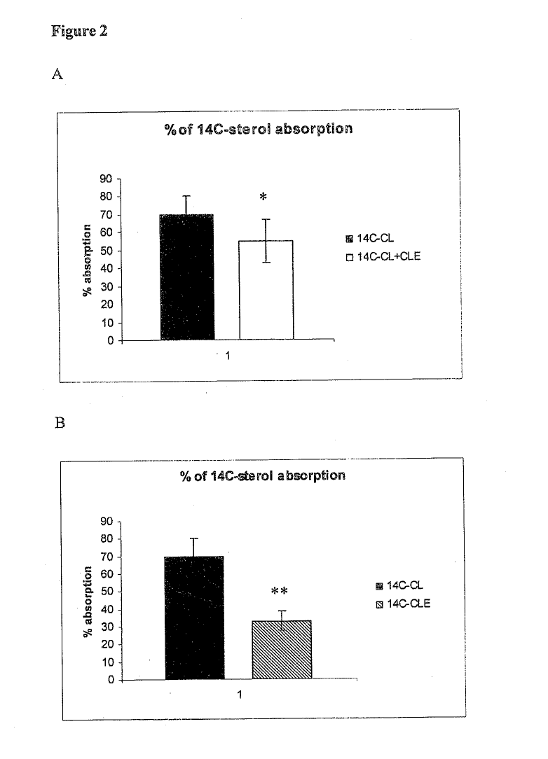

FIG. 2 shows intestinal absorption of sterols in mice using the plasma dual isotope ratio method. Panel A: addition of cholesteryl oleate to cholesterol containing milk (white column) decreased intestinal absorption of cholesterol in mice compared to absorption from milk containing cholesterol only (black column) Panel B: levels of absorption of cholesteryl oleate in the intestines of mice (hatched column) are lower than absorption of cholesterol (black column). Mean±SD, *p<0.05 for addition of cholesteryl oleate to cholesterol containing milk, **p<0.05 for cholesteryl oleate containing milk.

### Example 1

### Materials and Methods

### Chemicals

Sodium taurocholate, cholesterol, cholesteryl oleate, oleic acid, phosphatidylcholine in chloroform, sodium dodecyl sulfate (SDS), glucose solution and solvents were purchased from Sigma Aldrich Co. Dulbecco's modified Eagle's medium (DMEM), PBS, fetal bovine serum (FBS), nonessential amino acids (NEAA), penicillin-streptomycin and tripsin solutions were purchased from Gibco, NUNCLON flasks and 24 well plates were obtained from NUNC. Radiochemicals, [1,2-³H(N)]cholesterol (40-60 Ci/mmol) and cholesteryl oleate [Cholesteryl-1,2-3H(N)] (30-60 Ci/mmol) were obtained from Perkin Elmer Life Sciences. Opti-Phase HiSafe 2 liquid scintillant was purchased from Perkin Elmer-Wallac.

### Cell Culture

Human colon adenocarcinoma (Caco-2) cells were kindly provided by J T Rasmussen (Department of Molecular Biology Aarhus University C. F. Møllers Allé 3DK-8000 Aarhus CDenmark). Cells were routinely maintained in NUNCLON flasks in Dulbecco's Minimum Essential Medium (DMEM) supplemented with 4.5 g/l glucose, 10% heat-inactivated fetal bovine serum (FBS), 1% nonessential amino acid (NEAA), and 1% antibiotics (complete medium), as previously described (Hidalgo I J, Raub T J, Borchardt R T, Gastroenterology, (1989) March;96(3):736-49). Once the flasks reached 80% confluency, the cells were dispersed and seeded into 24-well plates at density 10⁵ cells/well in DMEM supplemented with 10% FBS and 1% NEAA. The cell monolayers were grown to confluence in 37° C. in a humidified atmosphere of 5% CO2 in air and allowed to differentiate for 15 days post-confluence with the culture medium replaced every other day.

### Preparatio of Artificial Micelles

Micelles were prepared according to the method described by Kirana (Kirana C, Rogers P F, Bennett L E, Abeywardena M Y, Patten G S, J Agric Food Chem. (2005) June 1;53(10:4623-7) with slight modifications. Briefly, for preparation of micellar solution of ³H labelled sterols, 14.8 kBq of [1,2-³H] labelled and 0.1 mM unlabelled cholesterol or cholesteryl oleate, respectively, 1 mM oleic acid, 5 mM phosphatidylcholine in chloroform, and 5 mM taurocholate salt were dissolved in ethanol and dried under nitrogen. An equivalent volume of serum-free DMEM was added and the suspension was sonicated three times for 1 min using ranson sonifier cell disruptor. The micelle solution was incubated overnight at 37° C. The solution was then centrifuged at 1000 g for 10 min followed by filtration through a 0.22 μm disposable syringe filter (PerkinElmer, Waltham, US-MA). The particle size of micelles was determined by dynamic light scattering (DLS) with use of the Malvern Zetasizer Nano series machine using latex 60nm Nanosphere Size Standard.

### Cholesterol and Cholesteryl Ester Absorption Assay

Monolayers were incubated at 37° C. for 45 min in micellar solutions containing [1,2-³H]cholesterol with or without unlabeled cholesteryl oleate. At the end of the incubation, medium containing micelles was collected and the cells were rinsed twice with cold PBS to remove unincorporated labeled cholesterol. The cells were lysed in 0.1% (w/v) SDS solution. A portion of the cell debris was mixed with Opti-Phase HiSafe 2 scintillant and the radioactivity was determined in a Microbeta Trilux Microplate Scintillation Analyzer (Perkin Elmer-Wallac) to estimate total cholesterol taken up by the cells. To investigate cholesteryl oleate uptake, cells were incubated with micelles containing [1,2-³H]cholesteryl oleate. The cells were analyzed as described above.

### Results

### Distribution of Micelle Particle Size.

In the artificially prepared micelles 100% had a diameter of 63-66 nm (data not shown).

### Effect of Cholesteryl Oleate on the Uptake of Micellar Cholesterol

It was postulated that the cholesteryl ester interfered with the uptake of cholesterol from the micelles. To address this, control cells were incubated with micelles containing labeled cholesterol. Another set of cells were incubated with the cholesterol micelles containing unlabeled cholesteryl oleate.

The results show that cells incubated with cholesterol micelles alone accumulated 30% more cholesterol compared to cells incubated with micelles containing both cholesterol and cholesteryl oleate (FIG. 1A). This figure shows that the inclusion of cholesteryl leate within the micelle decreases the uptake of micellar cholesterol by cultured CaCo-2 cells significantly (p<0.001).

### Uptake of Micellar Cholesteryl Oleate

To assess whether cholesteryl oleate was absorbed by CaCo-2 cells, cells were incubated with micelles containing either labeled cholesterol or ester.

Compared to cells incubated with micelles containing cholesterol, cells incubated with micelles containing cholesteryl oleate contained approximately 2-fold less labeled sterol (FIG. 1B). This suggests that uptake of the ester, as estimated by cell-associated radiolabeled sterol, was significantly less than that of cholesterol.

### Conclusion

Inclusion of cholesteryl oleate in cholesterol containing micelles decreases the uptake of micellar cholesterol by CaCo-2 cells significantly. Furthermore, uptake of cholesteryl oleate by CaCo-2 cells was significantly less than uptake of cholesterol. This indicates that cholesteryl oleate interferes with the uptake of micellar cholesterol. The results suggest that diet enrichment in cholesteryl esters may help to reduce intestinal cholesterol absorption resulting in lower blood cholesterol.

### Example 2

### Intestinal Absorption of Cholesterol and Cholesteryl Esters

Material and Method

Plasma Dual Isotope Ratio Method

The net intestinal absorption of cholester 1 and cholesteryl esters was measured using the plasma dual isotope ratio method described in Wang et al (Journal of Lipid Research, [2003] 44, 1042-1059).

This method is based on the simultaneous intragastric (IG) and intravenous (IV) administration of [³H]-cholesterol and [¹⁴C]-cholesterol, respectively, and measurement of plasma cholesterol isotope ratios at a set point in time. By definition, the IV [¹⁴C]-cholesterol dose corresponds to “100% absorption”, whereas the [³H]-cholesterol found in the blood reflects the absorption by the gastrointestinal tract. The method allows correction for post-absorptive cholesterol metabolism and for colonic handling of the malabsorbed labelled cholesterol by defining

$$\% Cholesterol absorption = \frac{Percent of IG dose {[}^{14} C ] \text{-} Ch per ml plasma}{Percent of IV dose {[}^{3} H ] \text{-} Ch per ml plasma} \times 100 \qquad ( 1 )$$

### Experimental Details

Fifteen wild type male mice (C57BL/6j), age of 5 weeks were randomly assigned into 3 groups. An amount of 2.5 μCi of [³H]-cholesterol was dissolved in 100 μl of phosphate buffered saline (PBS) and injected into the tail vein of non fasted and not-anesthetized animals. The animal were then given an oral bolus (IG) dose of either [¹⁴C]-cholesterol, [¹⁴C]-cholesterol cholesterol+unlabeled cholesteryl oleate (molar ratio 1:1) or [¹⁴C]-cholesteryl oleate dissolved in skimmed milk. Three days later, the mice were anesthetized (pentobarbital, IP) and bled by cardiac puncture into a tube containing heparin. The blood samples were centrifuged to pellet the blood cells and plasma. The percent of cholesterol absorption in plasma was calculated using (1).

### Results

As shown in FIG. 2A mice treated with [¹⁴C]-cholesterol and unlabeled cholesteryl oleate (molar ratio 1:1) showed a 12% reduction in cholesterol adsorption compared to [¹⁴C]-cholesterol treated mice (p<0.05).

Furthermore, FIG. 2B shows that [¹⁴C]-cholesteryl oleate treated mice showed a 50% reduction in cholesterol uptake compared to mice treated with [¹⁴C]-cholesterol. (p<0.001)

### Conclusion

Inclusion of cholesteryl oleate in cholesterol containing skimmed milk decreases the absorption of cholesterol by the mouse intestine. Furthermore, the level of intestinal absorption of cholesteryl oleate in mice is lower than absorption of cholesterol. The results indicate that cholesteryl oleate interferes with the absorption of micellar cholesterol. The results suggest that diets enriched in cholesteryl esters can help to reduce intestinal cholesterol absorption.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for reducing cholesterol absorption in an animal comprising administering to the animal a composition comprising an effective amount of at least one cholesterol ester.

2. The method according to claim 1 wherein the cholesterol ester is at least one of a cholesterol fatty acid ester, preferably a cholesterol fatty acid ester wherein the fatty acid is a C4 to C24 fatty acid.

3. The method according to claim 1, wherein the cholesterol ester comprises at least one of cholesteryl linoleate (C18:2), cholesteryl oleate (C18:1), cholesteryl stearate (C18:0), cholesteryl palmitate(C16:0), cholesteryl palmitoleate (C16:1), cholesteryl myristate (C14:0), cholesteryl laurate (C12:0) or cholesteryl caprate (C10:0).

4. The method according to claim 1, wherein the cholesterol ester is produced from at least one of egg, milk or meat.

5. The method according to claim 1, wherein the cholesterol ester is administered at a dosage of between about 0.001 g and about 1 Og per day, about 0.01 g and about 5 g per day, about 0.1 g and about 3 g per day.

6. The method according to claim 1, wherein the cholesterol ester is present in a foodstuff.

7. The method according to claim 6, wherein the foodstuff is selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

8. The method according to claim 6, wherein the cholesterol ester is added to the foodstuff or is produced in situ.

9. The method according to claim 1, wherein the cholesterol ester is formulated as a pharmaceutical composition comprising at least one pharmaceutically acceptable excipient, or carrier.

10. A method for reducing cholesterol absorption in an animal comprising administering
a. to the animal a composition comprising at least one lipid acyltransferase.

11. The method according to claim 10, wherein the composition further comprises at least one cholesterol ester.

12. The method according to claim 11, wherein the cholesterol ester is at least one of a cholesterol fatty acid ester, preferably a cholesterol fatty acid ester wherein the fatty
a. acid is a C4 to C25 fatty acid.

13. The method according to claim 11, wherein the cholesterol ester comprises at least one of cholesteryl linoleate (C18:2), cholesteryl oleate (C18:1), cholesteryl stearate (C18:0), cholesteryl palmitate(C16:0), cholesteryl palmitoleate (C16:1), cholesteryl myristate (C14:0), cholesteryl laurate (C12:0) or cholestery caprate (C10:0).

14. The method according to claim 10, wherein the cholesterol ester is administered at a dosage of between about 0.001 g and about 1 Og per day, about 0.01 g and about 5 g per day, about 0.1 g and about 3 g per day.

15. The method according to claim 10, wherein the lipid acyltransferase is present in a foodstuff.

16. The method according to claim 15, wherein the foodstuff is selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

17. The method according to claim 10, wherein the lipid acyltransferase is formulated as a pharmaceutical composition comprising at least one of a pharmaceutically acceptable diluent, excipient, or carrier.

18. The method according to claim 10, wherein the lipid acyltransferase is classified as an enzyme of class E.C. 2.3.1.x.

19. The method according to claim 10, wherein the enzyme has at least 5% activity when measured by the transferase assay described herein.

20. The method according to claim 1, which lowers the level of blood cholesterol in the animal.

21. The method according to claim 1, where the animal has hypercholesterolemia.

22. The method according to claim 1, where the animal is a human.

23. A composition comprising at least one cholesterol ester for use in therapy.

24. A composition comprising at least one cholesterol ester for use in reducing the level of blood cholesterol in an individual.

25. The composition for use according to claim 24, wherein the cholesterol ester is at least one of a cholesterol fatty acid ester, preferably a cholesterol fatty acid ester wherein the fatty acid is a C4 to C25 fatty acid.

26. The composition for use according to claim 24, wherein the cholesterol ester comprises at least one of cholesteryl linoleate (C18:2), cholesteryl oleate (C18:1), cholesteryl stearate (C18:0), cholesteryl palmitate(C16:0), cholesteryl palmitoleate (C16:1), cholesteryl myristate (C14:0), cholesteryl laurate (C12:0) or cholesteryl caprate (C10:0).

27. The composition for use according to claim 24, wherein the cholesterol ester is produced from at least one of egg, milk or meat.

28. The composition for use according to claim 24, wherein the cholesterol ester is administered at a dosage of between about 0.001 g and about 1 Ogper day, about 0.01 g and about 5 g per day, about 0.1 g and about 3 g per day.

29. The composition for use according to claim 24, wherein the composition comprises a foodstuff.

30. The composition for use according to claim 29, wherein the foodstuff is selected from selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

31. The composition for use according to claim 29, wherein the cholesterol ester is added to the foodstuff or is produced in situ.

32. The composition for use according to claim 24, wherein the cholesterol ester is formulated as a pharmaceutical composition comprising at least one pharmaceutically acceptable excipient, or carrier.

33. A composition comprising at least one lipid acyltransferase for use in reducing the level of blood cholesterol in an individual.

34. The composition for use according to claim 33, wherein the composition further comprises at least one cholesterol ester.

35. The composition for use according to claim 34, wherein the cholesterol ester is at leastone of a cholesterol fatty acid ester, preferably a cholesterol fatty acid ester wherein the fatty acid is a C4 to C25 fatty acid.

36. The composition for use according to claim 34, wherein the cholesterol ester comprises at least one of cholesteryl linoleate (C8:2), cholesteryl oleate (C18:1), cholesteryl stearate (C18:0), cholesteryl palmi tate(C16:0), cholesteryl palmitoleate (C16:1), cholesteryl myristate (C14:0), cholesteryl laurate (C12:0) or cholesteryl caprate (C10:0).

37. The composition for use according to claim 34, wherein the cholesterol ester is produced from at least one of egg, milk or meat.

38. The composition for use according to claim 34, wherein the cholesterol ester is administered at a dosage of between about 0.001 g and about 10 g per day, about 0.01 g and about 5 g per day, about 0.1 g and about 3 g per day.

39. The composition for use according to claim 33, wherein the composition comprises a foodstuff.

40. The composition for use according to claim 39, wherein the foodstuff is selected from selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

41. The composition for use according to claim 33, wherein the lipid acyltransferase is classified as an enzyme of class E.C. 2.3.1.x.

42. The composition for use according to claim 33, wherein the enzyme has at least 5% activity when measured by the transferase assay described herein.

43. The composition for use according to claim 24, wherein the individual has hypercholesterolemia.

44. A pharmaceutical composition comprising at least one cholesterol ester and at least one of a pharmaceutically acceptable diluent, excipient or carrier.

45. The pharmaceutical composition of claim 44 further comprising at least one lipid acyltransferase.

46. The pharmaceutical composition according to claim 44, wherein the cholesterol ester is at least one of a cholesterol fatty acid ester, preferably a cholesterol fatty acid ester wherein the fatty acid is a C4 to C24 fatty acid.

47. The pharmaceutical composition according to claim 44, wherein the cholesterol ester comprises at least one of cholesteryl linoleate (C18:2), cholesteryl oleate (C18:1), cholesteryl stearate (C18:0), cholesteryl palmitate(C16:0), cholesteryl palmitoleate (C16:1), cholesteryl myristate (C14:0), cholesteryl laurate (C12:0) or cholesteryl caprate (C10:0).

48. The pharmaceutical composition according to claim 44, wherein the lipid acyltransferase is classified as an enzyme of class E.C. 2.3.1.x.

49. The pharmaceutical composition according to claim 44, wherein the enzyme has at least 5% activity when measured by the transferase assay described herein.

50. A foodstuff comprising at least one exogenously produced cholesterol ester.

51. The foodstuff of claim 50 further comprising at least one lipid acyltransferase.

52. The foodstuff according to claim 50, wherein the cholesterol ester is at least one of a cholesterol fatty acid ester, preferably a cholesterol fatty acid ester wherein the fatty acid is a C4 to C25 fatty acid.

53. The foodstuff according to claim 50, wherein the cholesterol ester comprises at least one of cholesteryl linoleate (C18:2), cholesteryl oleate (C18:1), cholesteryl stearate (C18:0), cholesteryl palmitate(C16:0), cholesteryl palmitoleate (C16:1), cholesteryl myristate (C14:0), cholesteryl laurate (C12:0) or cholesteryl caprate (C10:0).

54. The foodstuff according to claim 53, wherein the foodstuff is selected from one or more of: eggs, egg-based products, including but not limited to mayonnaise, salad dressings, sauces, ice creams, egg powder, modified egg yolk and products made therefrom;
baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

55. The foodstuff according to claim 50, wherein the lipid acyltransferase is classified as an enzyme of class E.C. 2.3.1.x.

56. The foodstuff according to claim 50, wherein the enzyme has at least 5% activity when measured by the transferase assay described herein.

57. (canceled)

58. (canceled)

59. (canceled)

60. (Canceled)

61. The method according to claim 11, wherein the cholesterol ester is produced from at least one of egg, milk or meat.
